(19) United States
(12) Patent Application Publication
(10) Pub. No.: US 2007/0031526 A1
(43) Pub. Date: Feb. 8, 2007

(54) Topical Deodorant Compositions Based on Hydroxycitric Acid

(75) Inventor: Shyam K Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, Scottsdale, AZ (US)

(21) Appl. No.: 11/161,511

(22) Filed: Aug. 5, 2005

(51) Int. Cl. A61K 36/185; A61K 31/7008; A61K 31/555; A61K 31/522; A61K 31/4166; A61K 31/19; A61K 31/205

(52) U.S. Cl. 424/777; 514/184; 514/55; 514/263.32; 514/574; 514/356; 514/554; 514/62; 514/389; 514/350

(57) ABSTRACT

This invention relates to the use of Hydroxycitric acid and its derivatives in cosmetic and pharmaceutical compositions for reducing body malodor.

The enhancement of physical appearance occupies greater focus in human life than nearly all other daily life-related concerns combined. There are far more consumer products available for the beautification of human body than for the treatment of human ailments. The improvement of body appearance and body odor (malodor) is a growing, multibillion-dollar industry encompassing cosmetic, nutraceutical, pharmaceutical, and physical therapy disciplines. The consumer attention is focused on newest miracle ingredient for body odor control compositions.

The body odor (malodor) is known to be generated from several factors that include personal hygiene, dietary habits, and decomposition (by bacteria and fungi) of human sweat (eccrine and apocrine secretions).

The control of body odor can be accomplished in several ways that include: (1) the plugging of sweat pores with compositions that contain various aluminum salts, such as alum, aluminum chlorohydroxide, aluminum chloride, aluminum sulfate, and aluminum zirconium chlorohydroxide. Such compositions merely plug the sweat gland openings, thus reducing the amount of sweat on skin surface. However, this practice reduce body's ability to combat heat stress; (2) the killing of skin surface bacteria and fungi with antibacterial agents, such as triclocarban, triclosan, various zinc derivatives of fatty acids, and usnic acid. However, this practice is well known to result in the imbalance of good versus bad bacteria on skin surface; (3) the trapping of odor causing molecules by odor trap compositions, such as “Ordenone”; and (4) the masking of malodor by the use of fragrances and botanical extracts in deodorant compositions. However, the last two methodologies do not actually reduce either the biological cause or the composition of malodor constituents. These aspects have been further discussed by Lemoine et al (U.S. Patent application ser no. 20050163737).

A perfect deodorant ingredient should be able to accomplish the following highly desirable attributes: (1) Reduction or elimination of “food” in sweat that bacteria and fungi feed upon to generate malodor chemicals, such as various lower molecular weight fatty acids, amines, and sulfides; (2) No reduction in the amount of natural sweat production; (3) No effect on the natural habitat of desirable skin surface bacteria; (4) No contribution to odor (these molecules themselves be odor free); and (5) Compatibility with other desirable ingredients commonly used in cosmetic preparations.

It has now been discovered, surprisingly and unexpectedly, that hydroxycitric acid and some of its derivatives provide deodorancy benefits for topical applications. These hydroxycitric acid derivatives (such as hydroxycitric acid salts, amine complexes, and esters) provide: (1) A reduction or elimination of “food”, such as triglycerides, in sweat that bacteria and fungi feed upon to generate malodor chemicals, such as various lower molecular weight fatty acids; (2) No reduction in the amount of natural sweat production; (3) No effect on the natural habitat of desirable skin surface bacteria; (4) No contribution to odor (these molecules are themselves odor free); and (5) Are compatible with other desirable ingredients commonly used in cosmetic preparations.

The hydroxycitric acid and its derivatives are able to penetrate from topical compositions, especially when formulated in a pH range of about 3.0 to 7.5, and reduce the amount of triglyceride exudates in sweat. It is hypothesized that this is caused by the inhibition of Citrate Lyase enzyme by Hydroxycitrate moiety.

Hydroxycitric acid based compositions have received much attention in recent years for obesity control and the management of lean body mass.

U.S. Patent Application number 20020187943 (Majeed et al.) discloses hydroxycitric acid, obtained from the extracts of Garcinia cambogia plant, to cause a reduction in total body weight and body mass index, a reduction in body fat, an increase in lean body mass, and a reduction in perceived appetite level when taken by oral administration route. Although this patent does disclose the application of synergistic combinations of hydroxycitric acid for body weight management, none of the compositions were claimed to reduce body malodor.

U.S. Pat. Nos. 6,395,296 and 6,160,172 (both to Balasubramanyam et al) relates to a new soluble double metal salt of group IA and II A of (−) hydroxycitric acid, process of preparing the same and its use in beverages and other food products without effecting their flavor and properties. This product with >98% purity can be used safely not only as a food supplement in various nutraceutical formulations and beverages but also for effecting obesity control. Although this patent does disclose the application of synergistic combinations of hydroxycitric acid with several ingredients for body weight management, none of the compositions were claimed to reduce body malodor.

U.S. Patent Application 20020187204 and U.S. Pat. No. 6,413,545 (Alviar et al.) discloses a diet composition for managing body weight including effective amounts of Garcinia cambogia extract (which contains Hydroxycitric acid as its main component), Gymnema sylvestre extract, chromium picolinate, vanadium compound, L-carnitine, and conjugated linoleic acid. The daily effective amounts are administered in three approximately equal doses in conjunction with the daily meals. Although this patent does disclose the application of synergistic combinations of hydroxycitric acid with several ingredients for body weight management, none of the compositions were claimed to reduce body malodor.

Hydroxycitric acid has been disclosed to control inflammation (U.S. Patent application ser no. 20050032901).

Other derivatives of hydroxycitric acid have also been disclosed, for example U.S. Pat. Nos. 4,028,397; 4,007,208; 4,006,166; 4,005,086; 3,994,927; 3,993,668; 3,993,667; 3,966,772 (all to Guthrie et al.), and U.S. Pat. No. 5,911,992 (Braswell et al.) for fat-burning orally-administered nutraceutical compositions.

The combinations of hydroxycitric acid with other ingredients to control body weight have been disclosed in U.S. Patent application ser. no. 20050013887, 20050008726, 20050008725, 20050008724, 20050008723, 20040014692, and 20050008722.

As can be noted from the above prior art citations that Hydroxycitric acid and its derivatives have found applications in body fat reduction, slimming, and body toning. No prior art disclosures are known that claim the unique topical body odor (malodor) control benefits of Hydroxycitric acid and its derivatives.

For the control of topical body odor (malodor) prior art knowledge is abundant. However, none of those disclosures comprise hydroxycitric acid and its derivatives. This is further illustrated below.

Several plant based compositions have been disclosed to control body odor, for example Hagura et al (U.S. Patent application ser. no. 20050100520), Kim (U.S. Patent application ser. no. 20050089488), Akiyama et al. (U.S. Patent application ser. no. 20040241133), and Hwa (U.S. Patent ser. no. 20050152867). These extracts are not known to contain hydroxycitric acid or its derivatives.

Microbicidal deodorant compositions include disclosures by Morita et al, U.S. Pat. No. 6,881,418; Hoshima et al, U.S. Pat. No. 6,555,102; and Bhakoo et al, U.S. Pat. No. 6,709,647.

Odor absorbing deodorant compositions include Ascione et al., U.S. Pat. No. 6,632,421; Watanabe et al, U.S. Pat. No. 6,551,582; and Withiam et al., U.S. Patent application ser. no. 20050063928.

Aluminum salts based deodorant compositions include Mayes et al, U.S. Pat. No. 6,713,051; and Scavone et al, U.S. Pat. No. 6,403,071.

I have discovered that Hydroxycitric acid (HCA) and its derivatives, such as it various salts, amine complexes, amides, and esters, provide unexpected topical deodorancy benefits. It is hypothesized that Hydroxycitrate moiety can penetrate skin and cause a reduction of triglycerides in sweat and body exudates by the inhibition of Citrate Lyase enzyme. This reduction of triglycerides thus results in decreased production of lower molecular weight fatty acids that cause malodor from bacterial action on skin surface. This hypothesis, even if not correct, does not alter the net benefits of malodor reduction by HCA derivatives claimed in the present invention.

I have also discovered a simple method by which derivatives of HCA with organic hetero-atom bases (also referred to as “organic base” henceforth) can be made in-situ for their inclusion in cosmetic or pharmaceutical compositions that provide body deodorant benefits. These compositions are made by simple acid-base ion-pair chemical reaction, as shown in Equation 1, between an organic acid (RCOOH) and an organic heteroatom base.
RCOOH+(R′₃N)=[RCOO]⁻+[R′₃NH]⁺ (Equation 1).

To illustrate the scope of this invention, Equation 2 shows the formation of ion-pair form of niacinamide hydroxycitrate, a derivative of HCA (an organic acid) with niacinamide (an organic hetero-atom base), in a water solution.
HCA+Niacinamide=Niacinamide Hydroxycitrate (Equation 2).

Multi-component derivatives of HCA with organic bases can also be made by the in-situ method by mixing the reacting components in proportionate molar quantities in water or a mixture of water and water-miscible organic solvent solution, as illustrated in Equation 3.
HCA+Niacinamide+Allantoin+Glucosamine=Niacinamide Hydroxycitrate+Allantoin Hydroxycitrate+Glucosamine Hydroxycitrate (Equation 3).

The ingredients of present invention can be formulated in various topical delivery systems. Such cosmetically or pharmaceutically acceptable delivery systems include liquid, serum, lotion, cream, gel, mask, spray, roll-on, stick, powder, aerosol, and anhydrous compositions. Such compositions can be pre-made, and HCA derivatives disclosed in the present invention can be added as a solution in an appropriate solvent, or added directly into such pre-made compositions. Also, the HCA derivatives of the present invention can also be added to already formulated products. For example, in a body spray composition that has already been made, the hydroxycitric acid based ingredients for malodor control can be added.

Additional ingredient, such as colorants, skin care additives, fragrances, antioxidants, preservatives, emollients, humectants, skin soothing agents, anti-inflammatory agents, botanical extracts, odor trapping agents, odor neutralizers, antibacterials, aluminum salts, aluminum zirconium salts, et cetera, can also be included.

The present disclosure also relates to a cosmetic process for treating human perspiration and human underarm odors using the deodorant composition disclosed herein.

For the purposes of the present disclosure, the term “deodorant composition” means any composition capable of reducing the flow of sweat and of at least one of masking, absorbing, improving and/or reducing the unpleasant odor resulting from the decomposition of human sweat by bacteria.

As used herein, the term “antiperspirant aluminum salt” means any salt or any aluminum complex that has the effect of reducing or limiting the flow of sweat. The aluminum may be, for example, chosen from aluminum halohydrates; aluminum zirconium halohydrates; and complexes of zirconium hydroxychloride and of aluminum hydroxychloride with an amino acid, such as those described in U.S. Pat. No. 3,792,068, which are commonly known as “ZAG complexes”. Among the aluminum salts that may be mentioned, for example, are aluminum chlorohydrate in activated or inactivated form, aluminum chlorohydrex, aluminum chlorohydrex polyethylene glycol complex, aluminum chlorohydrex propylene glycol complex, aluminum dichlorohydrate, aluminum dichlorohydrex polyethylene glycol complex, aluminum dichlorohydrex propylene glycol complex, aluminum sesquichloro-hydrate, aluminum sesquichlorohydrex polyethylene glycol complex, aluminum sesquichlorohydrex propylene glycol complex, and aluminum sulfate buffered with sodium aluminum lactate. Among the aluminum zirconium double salts that may be mentioned, for example, are aluminum zirconium octachlorohydrate, aluminum zirconium pentachloro-hydrate, aluminum zirconium tetrachlorohydrate, and aluminum zirconium trichlorohydrate. An example of an aluminum zirconium double salt is the product sold by the company Reheis under the name Reach AZP-908-SUF. The complexes of zirconium hydroxychloride and of aluminum hydroxychloride with an amino acid are generally known under the name ZAG (when the amino acid is glycine). Among these products, mention may be made of the aluminum zirconium octachlorohydrex glycine, aluminum zirconium pentachlorohydrex glycine, aluminum zirconium tetrachlorohydrex glycine, and aluminum zirconium trichlorohydrex glycine complexes.

The deodorant compositions according to the disclosure intended for cosmetic use may be in the form of lotions, creams or fluid gels distributed as an aerosol spray, in a pump-dispenser bottle or as a roll-on, in the form of thick creams distributed in tubes or a grille; in the form of wands (sticks), and may comprise in this regard the ingredients generally used in products of this type and well known to those skilled in the art.

The deodorant compositions according to the present disclosure intended for cosmetic use may comprise at least one aqueous phase. They may be formulated, for example, in a form chosen from aqueous lotions, water-in-oil emulsions, oil-in-water emulsions, and multiple emulsions (oil-in-water-in-oil and water-in-oil-in-water triple emulsions (such emulsions are known and described, for example, by C. F. Fox in “Cosmetics and Toiletries”, November 1986, Vol. 101, pages 101-112)).

The at least one aqueous phase of the disclosed composition comprises water and generally other water-soluble or water-miscible solvents. The water-soluble or water-miscible solvents may be chosen from short-chain monoalcohols, for example, monoalcohols of C.sub.1-C.sub.4, such as ethanol and isopropanol; diols and polyols, for example, ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether and sorbitol. For example, propylene glycol and glycerol may be used.

According to one embodiment of the present disclosure, the antiperspirant compositions may be anhydrous.

For the purposes of the disclosure, the term “anhydrous” means a composition whose content of free or added water is less than 3% and for example, whose content of added water is less than 1%, by weight relative to the total weight of the composition.

The deodorant cosmetic composition according to the present disclosure can comprise, at option, at least one additional deodorant active agent chosen from, for example, bacteriostatic agents and bactericidal agents such as 2,4,4′-trichloro-2′-hydroxydiphenyl ether (Triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (Triclocarban) and 3,7,11-trimethyldodeca-2,5,10-trienol (Farnesol); quaternary ammonium salts, for example, cetyltrimethylammonium salts and cetylpyridinium salts; chlorhexidine and salts; diglyceryl monocaprate, diglyceryl monolaurate and glyceryl monolaurate; and polyhexamethylene biguanide salts.

The compositions according to the disclosure can comprise, at option, an organic powder. Among the fillers that may be used according to the disclosure, mention may be made of organic powders. As used herein, the term “organic powder” means any solid that is insoluble in the medium at room temperature (25.degree. C.). The at least one organic powder that may be used in the composition of the disclosure, include, for example, polyamide particles and for example, those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; polymethyl methacrylate microspheres, sold under the name Microsphere M-100 by the company Matsumoto or under the name Covabead LH85 by the company Wackherr; ethylene-acrylate copolymer powders, for example, those sold under the name Flobeads by the company Sumitomo Seika Chemicals; expanded powders such as hollow microspheres and for example, microspheres formed from a terpolymer of vinylidene chloride, of acrylonitrile and of methacrylate and sold under the name Expancel by the company Kemanord Plast under the references 551 DE 12 (particle size of about 12 mu.m and density of 40 kg/m.sup.3), 551 DE 20 (particle size of about 30 .mu.m and a density of 65 kg/m.sup.3) and 551 DE 50 (particle size of about 40.mu.m), or the microspheres sold under the name Micropearl F 80 ED by the company Matsumoto; powders of natural organic materials such as starch powders, for example, of corn starch, wheat starch or rice starch, which may or may not be crosslinked, such as the starch powder crosslinked with octenylsuccinate anhydride, sold under the name Dry-Flo by the company National Starch; silicone resin microbeads such as those sold under the name Tospearl by the company Toshiba Silicone, such as Tospearl 240; amino acid powders such as the lauroyllysine powder sold under the name Amihope LL-11 by the company Ajinomoto; particles of wax microdispersion, which for example, have mean sizes of less than 1.mu.m and for example, range from 0.02.mu.m to 1.mu.m, and which consist essentially of a wax or a mixture of waxes, such as the products sold under the name Aquacer by the company Byk Cera, and for example, Aquacer 520 (mixture of synthetic and natural waxes), Aquacer 514 or 513 (polyethylene wax), Aquacer 511 (polymer wax), or such as the products sold under the name Jonwax 120 by the companyjohnson Polymer (mixture of polyethylene wax and paraffin wax) and under the name Ceraflour 961 by the company Byk Cera (micronized modified polyethylene wax); and mixtures thereof.

The cosmetic composition according to the disclosure may also comprise at least one cosmetic adjuvant chosen from waxes, softeners, antioxidants, opacifiers, stabilizers, moisturizers, vitamins, fragrances, bactericides, preserving agents, polymers, fragrances, thickeners, propellants, or any other ingredient usually used in cosmetics for this type of application. Needless to say, a person skilled in the art will take care to select this or these optional additional compound(s) such that the advantageous properties intrinsically associated with the cosmetic composition in accordance with the disclosure are not, and are not substantially, adversely affected by the envisaged addition(s).

The waxes may be chosen from animal, fossil, plant, mineral and synthetic waxes. Mention may be made, for example, to beeswaxes, carnauba wax, candelilla wax, sugar cane wax, Japan wax, ozokerites, montan wax, microcrystalline waxes, paraffins, and silicone waxes and resins.

The thickeners, which are, for example, nonionic, may be chosen from modified and unmodified guar gums and celluloses, such as hydroxypropyl guar gum and cetylhydroxyethylcellulose, silicas, such as Bentone Gel MIO sold by the company NL Industries, and Veegum Ultra sold by the company Polyplastic.

The amounts of these various constituents, i.e., optional cosmetic adjuvants, that may be present in the cosmetic composition according to the disclosure are those conventionally used in deodorant compositions.

The compositions according to the disclosure may also contain at least one other agent for structuring or gelling the at least one water-immiscible organic liquid phase of the composition chosen from linear solid fatty alcohols and waxes; fatty acids and salts thereof (stearic acid, sodium stearate or 12-hydroxystearic acid; dibenzylidene alditols (DBS); lanosterol, N-acylamino acid derivatives; di- and tricarboxylic acid derivatives, such as alkyl-N,N′-dialkylsuccin-amides (i.e., dodecyl-N,N′-dibutylsuccinamide); elastomeric polyorganosiloxanes such as, those described in International Patent Application No. WO 97/44010.

The composition according to the disclosure may also be pressurized and may be packaged in an aerosol device. The present disclosure is further related to an aerosol device comprising a container comprising a composition as defined above, at least one propellant and a device for distributing the aerosol composition. The at least one propellant generally used in products of this type, which are well known to those skilled in the art, are, for example, dimethyl ether (DME); volatile hydrocarbons such as n-butane, propane or isobutane, and mixtures thereof, optionally with at least one chlorohydrocarbon and/or fluorohydrocarbon; among the latter, mention may be made of the compounds sold by the company Dupont de Nemours under the names Freon® and Dymel®, and for example, monofluorotrichlorometh-ane, difluorodichloromethane, tetrafluorodichloroethane and 1,1-difluoroethane sold, for example, under the trade name Dymel 152 A by the company Dupont. Carbon dioxide, nitrous oxide, nitrogen or compressed air may also be used as the at least one propellant. The composition comprising the deodorant composition and the at least one propellant may be in the same compartment or in different compartments in the aerosol container. According to the disclosure, the concentration of propellant generally ranges from 5% to 95% by pressurized weight and for example, from 50% to 85%, by weight relative to the total weight of the pressurized composition. The distribution device, which forms a part of the aerosol device, generally comprises a distribution valve controlled by a distribution head, itself comprising a nozzle via which the aerosol composition is vaporized. The container comprising the pressurized composition may be opaque or transparent. It may be made of glass, of polymeric material or of metal, optionally coated with a coat of protective varnish.

The present disclosure further relates to a cosmetic process for treating human underarm odors comprising applying to the underarm area an effective amount of a composition as defined above.

### EXAMPLES

The following examples are presented to illustrate presently preferred practice thereof. As illustrations they are not intended to limit the scope of the invention. All quantities are in weight %.

### Example 1

Preparation of Hydroxycitric Acid from HCA Salts. Ingredients. (1) Tripotassium Hydroxycitrate Hydrate 34.0, (2) Deionized water 36.0, (3) Hydrochloric Acid (10 molar solution in water) 30.0. Procedure. Mix (1) and (2) with heating to a clear solution. Add (3) and mix. Upon cooling, potassium chloride precipitates out, which is then removed by filtration. A solution of hydroxycitric acid (20%) in water is thus obtained. This solution is useful for other compositions that may require hydroxycitric acid.

### Example 2

In-Situ Preparation of Niacin Dipotassium Hydroxycitrate from Niacin Hydrochloride and Tripotassium Hydroxycitrate. Ingredients. (1) Tripotassium Hydroxycitrate Hydrate 3.4 (2) Deionized Water 95.3, (3) Niacin Hydrochloride 1.3. Procedure. Mix (1) to (3). Niacin Dipotassium Hydroxycitrate (3.96%) is formed in situ. Potassium chloride, also formed in this reaction, is precipitated by the addition of ethanol, followed by filtration.

### Example 3

Niacinamide Hydroxycitrate Deodorant Spray. Ingredients. (1) Deionized Water 67.55 (2) Glycerin 2.0 (3) Niacinamide Hydroxycitrate 1.2 (4) Aloe vera 0.05 (5) Preservatives 1.0 (6) Zinc Gluconate 0.2 (7) Polysorbate-20 4.0 (8) Witch Hazel extract 24.0. Procedure: Mix all ingredients to a clear solution.

### Example 4

Niacinamide Hydroxycitrate Deodorant Cologne. Ingredients. (1) Deionized Water 67.55 (2) Glycerin 2.0 (3) Niacinamide Hydroxycitrate 1.2 (4) Aloe vera 0.05 (5) Preservatives 1.0 (6) Zinc Gluconate 0.2 (7) Polysorbate-20 4.0 (8) Alcohol SDA-40 20.0 (9) Fragrance 1.0 (10) Ordenone (odor trapping agent) 3.0. Procedure: Mix all ingredients to a clear solution.

### Example 5

Deodorant Aerosol. Ingredients. (1) Stearalkonium Bentonite 0.5 (2) (2) Aluminum chlorohydrate 7.0 (3) Niacinamide Hydroxycitrate 1.0 (4) C12-C15 Alkyl benzoate 3.0. (5) Cyclopentasiloxane 6.5 (6) Triethyl citrate 1.0 (7) Isopropyl palmitate 1.0 (8) Isobutane 80.0. Procedure. Mix (1) to (7) and homogenize. Fill in aerosol containers and fill (8).

### Example 6

Deodorant Sticks. Ingredients. Ingredients. (1) Cyclopentasiloxane 32.0 (2) Hexyldecyl stearate 15.0 (3) PPG-14 butyl ether 5.0 (4) Hydrogenated castor oil 6.0 (5) Cetearyl alcohol/Ceteareth-30 15.0 (6) Talc 8.0 (7) PEG-8 distearate 2.0 (8) C12-15 alkyl benzoate 15.0 (9) Zinc Hydroxycitrate 2.0. Procedure. Cyclopentasiloxane was heated to 65.degree. C. The other ingredients were added (one by one) while keeping the temperature at 65.degree.-70.degree. C. The mixture was homogenized for 15 minutes. The resulting mixture was cooled to about 55.degree. C. (a few degrees Celsius above the thickening of the mixture) and was cast into sticks. The sticks were placed at 4.degree. C. for 30 minutes.

### Example 7

Niacinamide Hydroxycitrate Deodorant Spray Test Control Composition. Ingredients. (1) Deionized Water 68.75 (2) Glycerin 2.0 (3) Niacinamide Hydroxycitrate 0.0 (4) Aloe vera 0.05 (5) Preservatives 1.0 (6) Zinc Gluconate 0.2 (7) Polysorbate-20 4.0 (8) Witch Hazel extract 24.0. Procedure: Mix all ingredients to a clear solution.

Consumer Test Protocol for Odor Evaluation.

The composition of Example 3 and Example 7 were evaluated by a consumer panel of 20 people having a varying degree of body malodor. The composition of Example 3 (with Niacinamide Hydroxycitrate) was applied to armpit 1 and composition of Example 7 (without Niacinamide Hydroxycitrate) was applied to armpit 2. The evaluations were performed by consumer by a self-evaluation method for the intensity of malodor after 8 hours of application. The deodorancy efficacy was graded on the basis of the intensity of body odor in the armpit area on a scale of 1 to 10 (1=imperceptible odor to 10=extremely strong odor).

The data are tabulated below. The data analysis shows a greater than 38% reduction of malodor.

| | Armpit | Armpit |
|---|---|---|
| Panelist # | 1 | 2 |
| 1 | 5 | 8 |
| 2 | 4 | 7 |
| 3 | 7 | 9 |
| 4 | 3 | 6 |
| 5 | 3 | 5 |
| 6 | 6 | 8 |
| 7 | 4 | 6 |
| 8 | 5 | 7 |
| 9 | 5 | 6 |
| 10 | 3 | 4 |
| 11 | 2 | 3 |
| 12 | 2 | 4 |
| 11 | 5 | 8 |
| 12 | 7 | 8 |
| 13 | 8 | 8 |
| 14 | 5 | 7 |
| 15 | 4 | 5 |
| 16 | 6 | 7 |
| 17 | 3 | 5 |
| 18 | 6 | 8 |
| 19 | 7 | 8 |
| 20 | 4 | 7 |
| Total Score | 104 | 144 |
| Average | 5.2 | 7.2 |
| % OdorReduction | 38.46 | |

What is claimed is:

1. A composition to control topical body malodor, which includes a hydroxycitric acid derivative.

2. A composition according to claim 1, wherein hydroxycitric acid derivative is Hydroxycitric acid.

3. A composition according to claim 1, wherein hydroxycitric acid derivative is an extract of Garcinia Cambogia.

4. A composition according to claim 1, wherein hydroxycitric acid derivative is Garcinia Lactone.

5. A composition according to claim 1, wherein hydroxycitric acid derivative is Garcinia Acid.

6. A composition according to claim 1, wherein hydroxycitric acid derivative is Niacinamide Hydroxycitrate.

7. A composition according to claim 1, wherein hydroxycitric acid derivative is Pyridoxine Hydroxycitrate.

8. A composition according to claim 1, wherein hydroxycitric acid derivative is Zinc Hydroxycitrate.

9. A composition according to claim 1, wherein hydroxycitric acid derivative is selected from Sodium Hydroxycitrate, Potassium Hydroxycitrate, Calcium Hydroxycitrate, Magnesium Hydroxycitrate, or combinations thereof.

10. A composition according to claim 1, wherein hydroxycitric acid derivative is selected from Allantoin Hydroxycitrate, Glucosamine Hydroxycitrate, Creatine Hydroxycitrate, Carnitine Hydroxycitrate, Chitosan Hydroxycitrate, Niacin Hydroxycitrate, Benzyl Niacin Hydroxycitrate, Methyl Niacin Hydroxycitrate, Caffeine Hydroxycitrate, Aminophylline Hydroxycitrate, Chromium picolinate Hydroxycitrate, Phaseolamin Hydroxycitrate, Theophylline Hydroxycitrate, Theobromine Hydroxycitrate, Synephrine Hydroxycitrate, Hordenine Hydroxycitrate, Octopamine Hydroxycitrate, Tyramine Hydroxycitrate, N-Methyltyramine Hydroxycitrate, Matrine Hyd roxycitrate, Oxymatrine Hydroxycitrate, and combinations thereof.

11. A composition according to claim 1, wherein Hydroxycitric acid derivative is selected from Hydroxycitric acid esters.

12. A composition according to claim 1, wherein Hydroxycitric acid derivative is selected from Hydroxycitric acid amides.

13. A composition according to claim 1, wherein a cosmetically acceptable topical delivery system is included.

14. A composition according to claim 1, wherein Hydroxycitric acid derivative can be from 0.0001 to 60 weight % of the total composition.

15. A composition according to claim 1, wherein additional skin beneficial ingredients, such as anti-oxidants, surfactants, cleansing agents, bleaching agents, antiperspirants, vitamins, hormones, minerals, plant extracts, skin whitening agents, anti-inflammatory agents, concentrates of plant extracts, emollients, moisturizers, skin protectants, humectants, silicones, skin soothing ingredients, sun screens, analgesics, anesthetics, colorants, perfumes, and like can be added to the formulation.

16. A composition according to claim 11, wherein Hydroxycitric acid ester is selected from 1 to 20 carbon chain alkyl alcohol esters of Hydroxycitric acid.

17. The compositions according to claim 13, wherein the cosmetically acceptable delivery system can be traditional water and oil emulsions, suspensions, solutions, gels, colloids, and anhydrous systems, and combinations thereof.
